(19) United States
(12) Patent Application Publication
(10) Pub. No.: US 2003/0099645 A1
(43) Pub. Date: May 29, 2003

(54) Naturally occuring IgM antibodies that bind to membrane receptors on lymphocytes

(76) Inventor: Peter Isaac Lobo, Charlottesville, VA (US)

Correspondence Address: Peter I. Lobo, 348 Keywest Drive, Charlottesville, VA, 22911, US

(21) Appl. No.: 10/292,002

(22) Filed: Nov. 7, 2002

Related U.S. Application Data
(63) Continuation-in-part of application No. 09/684,813, filed on Oct. 10, 2000.

(51) Int. Cl. A61K 39/395

(52) U.S. Cl. 424/144.1

(57) ABSTRACT

Human and animal serum contains naturally occurring autoantibodies that develop at birth in absence of deliberate immunization. These antibodies are predominantly of IgM isotype but can include all immunoglobulin isotypes such as IgD, IgA and IgG. Here we describe IgM anti-lymphocyte autoantibodies and show that these antibodies are heterogenous with some antibodies binding to chemokine receptors such as CCR5 and CXCR4 and others binding to other T cell receptors including CD3. IgM antibodies inhibit HIV-1 from infecting cells, inhibit chemokine from binding to receptors and inhibit activation and proliferation of T lymphocytes. IgM antibodies that bind to lymphocyte also bind to other leucocytes and other cells such as cancer cells and endothelial cells. The inventor claims that naturally occurring anti-lymphocyte antibodies of all immunoglobulin isotypes inhibit viral infections, cancer and several inflammatory states by binding to chemokine receptors and other cellular receptors that activate cells or promote viral entry.

This application is a Continuation-In-Part of U.S. patent application Ser. No. 09/684,813, filed Oct. 10, 2000, which claims priority to patent application Ser. No. 09/439,690 filed Nov. 14, 1999 and to U.S. Provisional Patent Application Serial No. 60/108,937, filed Nov. 18, 1998. The entirety of these applications is incorporated herein by reference.

### BACKGROUND OF THE INVENTION

1. Field of the Invention

The present invention relates generally to naturally occurring IgM anti-lymphocyte antibodies and, more particularly, to a method of inhibiting disease progression through use of these antibodies.

2. Discussion of the Background

Normal humans and animals have naturally occurring IgM auto-antibodies (referred to as IgM NAA), which are present at birth and produced in the absence of deliberate immunization with the target antigen. Prior art has clearly demonstrated that IgM NAA are mostly polyreactive in that a single monoclonal IgM NAA can recognize several closely similar self antigens, which possess a unique but distinct set of epitope specificities. The nature of this polyreactivity is best exemplified by rheumatoid factor, which is an IgM NAA that recognizes and binds to the Fc region of different self and non-self IgG but does not bind to other glycoproteins or self nucleo-proteins. The antigen binding site of IgM NAA are in general encoded by germline genes, which are subjected to no or minimal mutation and this characteristic is responsible for the polyreactivity of these antibodies. Conversely, genes encoding the antigen binding site of antibodies, produced in response (after immunization) to a foreign antigen are hypermutated and this genetic characteristic renders these antibodies highly specific with high binding affinity. Hence, the polyreactivity and low binding affinity of IgM NAA resulting form their genetic makeup distinguishes these antibodies from the conventional antibodies produced after deliberate immunization. Prior art has used antibodies, typically produces after immunization, and with high binding affinity and with high specificity, to protect against infections or to inhibit immune mediated disorders. The current invention is novel in that the antibodies used are naturally occurring.

Normal human and animals have in their blood low levels of circulating naturally occurring IgM antibodies that bind to their own leukocytes such as, for example, B and T lymphocytes, without causing cell lysis at 37° C. Such IgM antibodies are, therefore, referred to as “IgM anti-lymphocyte autoantibodies.” These IgM anti-lymphocyte antibodies bind to macrophages, neutrophils, endothelial cells and malignant cells and furthermore bind to allogenic cells in addition to autologous leukocytes. Both, animal IgM anti-lymphocyte NAA (mouse, rat, goat, horse, rabbit) and human IgM anti-lymphocyte NAA bind to the same human cells. Hence, in this application, IgM anti-lymphocyte auto-antibodies (whether human or animal) will be referred to as IgM anti-lymphocyte or leucocyte antibodies or autoantibodies, i.e. autoantibodies or antibodies will be used interchangeably. Very little is known about the leukocyte or lymphocyte antigens or receptors that bind to IgM autoantibodies.

Levels of such anti-leukocyte antibodies increase during inflammatory states, including autoimmune diseases and infectious diseases such as, for example, systemic lupus erythematosus (“SLE”), sarcoidosis, HIV-1, malaria, Epstein-Barr virus (“EBV”) and cytomegalovirus (“CMV”). Individuals with asymptomatic HIV-1, therefore, have high levels of IgM anti-leukocyte autoantibodies.

The inventor's studies show, however, that chemokine receptors are one of the cell membrane receptors that bind to these IgM autoantibodies and that, through this mechanism, such IgM autoantibodies inhibit HIV-1 from infecting cells. The inventor's studies also show that IgM autoantibodies that bind to lymphocyte receptors are heterogeneous and that only some of these antibodies have the ability to inhibit HIV-1 from infecting cells. Levels of IgM antibodies that inhibit HIV-1 from infecting cells are very low or deficient in patients with AIDS. Thus, while individuals with asymptomatic HIV-1 infection have increased levels of IgM autoantibodies that inhibit HIV-1 infectivity, these levels, however, significantly decrease as the disease progress to AIDS. Total serum IgM does not decrease, however, as the disease progresses to AIDS. The inventor's studies also show that IgM binds to the CD3 antigen on lymphocytes. Accordingly, IgM NAA inhibits lymphocyte activation and proliferation by binding to both or either the CD3 receptor and the chemokine receptors. More information on IgM NAA and IgM anti lymphocyte antibodies are reviewed in Lacroix-Desmazes S., et al J of Immunol Methods 216: 117-137, 1998 and the material in this reference is incorporated herein by reference.

The inventor will now briefly provide a summary of chemokines and chemokine receptors. Details on this subject are described by Olson and Ley, Amer. J Physiol Regulatory Integrative Comp Physiology 283: R7-R28, 2002; by Gerard and Rollins, Nature Immunol 2: 108-115, 2001; and by Onuffer and Horuk, Trends in Pharmacological Sciences 23: 459-467 and the material in these 3 references is incorporated herein by reference.

The known chemokine system in humans comprises, approximately 50 different chemokines and about 20 G-protein coupled chemokine receptors. The chemokine system has several characteristics (i) Most chemokines are secreted but some e.g. fractalkine are expressed on the cell surface. (ii) Chemokines are subdivided into CC, CXC, or CX₃C groups based on the number of amino acids between the first two cysteines (iii) Certain chemokines bind only one receptor e.g. CXCR4 with SDF-1 and CXCR5 with BCA-1 while other receptors can bind to several chemokines e.g. CXCR3 binds to IP-10, Mig and I-TAC. Similarly, a single chemokine can bind to several receptors e.g. RANTES will bind to CCR1, CCR3 and CCR5 with high affinity. This has led many in the field to suggest that the chemokine system was rife with redundancy. However, there are certain exceptions as lack of CXCR4 receptor expression is associated with abnormal embryogenesis and organogenesis. In addition, different chemokine receptors expressed on the same cell can induce specific signals, thus indicating that receptors are coupled to distinct intracellular pathways. (iv) Certain chemokines (and their respective receptors), important for normal homeostatic trafficking (e.g. BCA-1, which is involved with normal migration of lymphocytes to lymph nodes), are constitutively expressed while inflammatory chemokines (and their receptors) are induced on leucocytes and other cells e.g. endothelial cells, only under specific conditions, typically by inflammatory chemokines e.g. IL-1 or TNF-produced by macrophages or activated T lymphocytes. (v.) Chemokine receptors are expressed on many different cells including leucocytes, endothelial cells, smooth muscle cells, and epithelial cells and neuronal cells and these cells can also secrete chemokines.

Chemokines play a prominent role in leucocyte trafficking that occurs with several inflammatory processes as diverse as multiple sclerosis, rheumatoid arthritis, systemic lupus erythematosus, vasculitis, allograft and xenograft rejections, acute and chronic bacterial and viral infections, asthma, colitis, psoriasis, atherosclerosis, hypertension, ischaemia-reperfusion and inflammation associated with neoplasia. Additionally, chemokines play a role in other non-inflammatory processes e.g. organo-genesis, hematopoiesis, and neuronal communication with microglia and with angiogenesis. The pivotal role played by chemokines in some of these disorders is illustrated by the observation that (a) specific deficiency of CXCR4 is associated with abnormal organo-genesis and (b) individuals with a homozygous defect in CCR5 are protected from allograft rejections and asthma. The participation of the chemokine system in inflammatory processes involves leucocyte trafficking as well as leucocyte activation and immune cell differentiation. For example, chemokines induce neutrophils to increase integrin expression, neutrophil degranulation and super oxide formation. Similarly, the chemokine system is involved in tissue-specific homing of lymphocyte subsets to lymphoid organs where lymphocytes get activated and start differentiating (see Olson and Ley reference).

Of particular significance is the finding that chemokine receptors i.e. predominantly CXCR4 and CCR5 act as co-receptors for the entry of HIV-1 virus into cells. The X4 HIV-1 virus uses the CXCR4 receptor while the R5 HIV-1 virus uses the CCR5 receptor. It has become abundantly clear that viral entry through chemokine receptors is of prime importance in influencing viral replication and disease progression after an HIV-1 infection. For example, individuals with genetic defects in the CCR5 receptor have been associated with a prolonged latency period after HIV-1 infection i.e. a slower progression of HIV-1 to AIDS.

Researchers and pharmaceutical companies have been looking into strategies to block or inactivate specific chemokine receptors in an effort to inhibit inflammatory processes that induce disease processes and to inhibit HIV-1 entry into cells. Some of these include use of peptides and IgG monoclonal antibodies that will bind to specific chemokine receptors. Such strategies, however, have not as yet been shown to be effective.

### SUMMARY OF THE INVENTION

Normal humans and animals have naturally occurring IgM autoantibodies (referred to as IgM NAA), which are present at birth and produced in the absence of deliberate immunization with the target antigen. IgM NAA are distinct from antibodies produced after immunization with foreign antigen in that the antigen binding site of NAA are encoded by germ line genes, which undergo minimal or no mutation. As a result, IgM NAA lack specificity and have low binding affinity. IgM NAA are mostly polyreactive in that a single IgM monoclonal antibody can recognize several closely similar self-antigens, which possess a unique but distinct set of epitope specificities. While the presence of IgM anti-lymphocyte NAA has previously been described, there is no prior art identifying the glycoprotein lymphocyte receptors targeted by IgM, nor is there prior art showing that IgM anti-lymphocyte NAA can alter cell function or inhibit viral infectivity of cells.

In the present invention, applicant has discovered that some of the IgM anti-lymphocyte NAA obtained from normal human sera bind to chemokine receptors and specifically inhibit binding of chemokines to their receptors, enhance or inhibit chemotaxis and inhibit HIV-1 from infecting cells. IgM autoantibodies that inhibit HIV-I from infecting cells are depleted in patients with AIDS but not in asymptomatic HIV-1 infected individuals or in normal individuals. Accordingly, the inventor believes that IgM NAA inhibits HIV-1 infectivity by “blocking” HIV-1 entry through the chemokine receptor as well as by inhibiting lymphocyte activation by binding to chemokine receptors and other receptors e.g. CD3 (see below).

Moreover, IgM anti-lymphocyte NAA are a heterogenous group of several different antibodies that bind to chemokine and other non-chemokine receptors on the lymphocyte. Such non-chemokine receptors include glycoprotein and glycolipid receptors. These IgM anti-lymphocyte NAA have low binding affinity and do not lyse normal cells in the presence of complement at body temperature (i.e. 37° C.). Applicant, in this invention has discovered that these polyreactive IgM anti-lymphocyte NAA bind to the same or closely similar lymphocyte receptors (e.g. chemokine or non-chemokine receptors) that are also present on other leucocytes (i.e. neutrophils, eosinophils, and macrophages), endothelial cells and malignant cells (both lymphoid and non-lymphoid). In the present invention, applicant also demonstrates that IgM anti-lymphocyte NAA binds to a non-chemokine receptor, identified as CD3, and further shows that naturally occurring IgM with anti-CD3 and anti-chemokine receptor activity inhibits lymphocyte activation and proliferation.

The inventor has observed that human kidney transplant recipients, who have high levels of IgM reactive to their donor lymphocytes rarely, have problems with rejections. Applicant, in this invention, believes that protection against rejection is mediated by the inhibitory effect of IgM on autologous leucocytes and donor endothelial cells. High level binding of recipient IgM to donor lymphocytes is also associated with similar level of IgM binding to recipient leucocytes and donor endothelial cells. Recipient IgM would thus protect against rejection by inhibiting leucocyte migration as well as by inhibiting activation of autologous lymphocytes e.g. through binding to CD3 and chemokine receptors.

Finally, the inventor has observed increased cell death and lysis of malignant cells, (but not normal cells) at 37° C. in presence of normal IgM anti-lymphocyte NAA. The inventor believes that these antibodies also protect against malignancy.

Accordingly, one object of the present invention is to provide a method of inhibiting disease processes involving and/or mediated by chemokine and non-chemokine receptors through use of IgM anti-lymphocyte NAA.

The above and other objects, advantages and features of the present invention will become more apparent from the following detailed description of the presently preferred embodiments, when considered in conjunction with the figures, and to the appended claims.

### BRIEF DESCRIPTION OF THE DRAWINGS

FIG. 1A is a flow cytometry histogram depicting binding of Normal IgM, HIV IgM and AIDS IgM to Sup T-1 cells.

FIG. 1B is a graph depicting binding of Normal IgM, HIV IgM and AIDS IgM to Ghost CXCR4 cells.

FIG. 1C is a flow cytometry dot plot showing lymphocytes and neutrophils separated by size and derived from human blood.

FIG. 1D is a flow cytometry histogram depicting binding of Normal IgM to human T lymphocyte derived from peripheral blood cells.

FIG. 1E is a flow cytometry histogram depicting binding of AIDS IgM to human neutrophils derived from peripheral blood cells.

FIG. 2 is a flow cytometry dot plot depicting the binding (or lack of binding) of normal human IgM to different solubilized cell membrane receptors (derived from U937 or Jurkat cells) to form immune complexes that were then immunoprecipitated with agarose beads covalently linked to goat anti-human IgM.

FIG. 3A is a graph depicting affinity purified Accurate Normal Pooled IgM and monoclonal IgM (CK15) inhibition of I¹²⁵ RANTES binding to CCR5 present in U373-MAGI-CCR5E membrane proteins as quantified by liquid scintillation.

FIG. 3B is a Western blot assay depicting affinity purified Accurate Normal Pooled IgM inhibition of I¹²⁵ RANTES binding to CCR5 present in U373-MAGI-CCR5E membrane proteins.

FIGS. 4A and 4B are flow cytometry histograms depicting normal, HIV and AIDS IgM inhibition of biotin-labeled SDF-1α binding to CXCR4 on intact Sup T-1 cells.

FIG. 4C is another graph depicting the effect of different doses of normal pooled IgM on inhibition of boitin labeled SDF-1α binding to CXCR4 present on intact Sup T-1 cells.

FIG. 4D is a flow cytometry dot plot depicting a dose dependent inhibition by normal IgM of RANTES (biotin labeled) binding to intact 1L-2 activated human lymphocytes.

FIG. 4E is a flow cytometry histogram depicting inhibition by normal pooled IgM (but not by normal pooled IgG) of biotin labeled RANTES binding to intact U373-MAG1-CCR5E transfectants.

FIGS. 5A-5B indicate the effect of IgM on chemotaxis and intracytosolic Ca⁺².

FIGS. 6A, 6B and 6C are graphs depicting a dose dependent effect of Normal, HIV and AIDS IgM on HIV-1 R5 and X4 infectivity of Ghost cells.

FIG. 6D is a graph depicting the protective effect of normal human IgM on HIV IIIB (X4 virus) infection of human PBL introduced into the peritoneal cavity of SCID mice.

FIG. 7A is a flow cytometry do plot to depict the quantitative binding of kidney transplant recipient serum IgM (i.e. quantified as No, Low or High) to donor T lymphocytes (CD3 positive) in the absence of recipient serum (upper panels, negative controls) and in the presence of recipient serum (lower panels).

FIG. 7B is a table correlating the incidence of acute kidney rejections and loss of kidney grafts (at 1 year) with quantity of IgM bound to donor T lymphocytes.

### DESCRIPTION OF THE PREFERRED EMBODIMENTS

To achieve the foregoing and other objects, and in accordance with the purpose of the present invention as embodied and broadly described herein, the present invention relates to the expression, stimulation and administration of IgM receptor-binding antibodies to address viral infections and disease states induced thereby.

Prior art has shown that IgM auto antibodies present in the blood of normal uninfected individuals and in newborns bind to extracellular receptors present on lymphocytes. There is prior art to also show that IgM auto antibodies to lymphocytes which are present in low levels in normals, increase in various infectious states (including HIV), auto immune disorders, and inflammatory disorders. These IgM antibodies are heterogenous and bind to several unidentified membrane receptors. However, there is prior art to show that IgM auto antibodies bind to glycosphingolipid and phospholipid membrane antigens on the lymphocyte membrane. These IgM auto antibodies do not damage normal cells at 37° C. as at that temperature they have a low binding affinity and cannot activate complement.

According to the present invention, IgM anti-lymphocyte auto antibodies present in normal sera bind to chemokine receptors, for example, CXCR4, CCR5, CCR3 and CCR2b and other non-chemokine lymphocyte-surface receptors e.g. the CD3 antigen. The inventor also shows that there is a subset of IgM anti-lymphocyte receptor auto antibody that inhibits HIV-1 from infecting cells and this IgM subset is depleted in patients with AIDS.

While not wishing to be bound to a specific theory, the inventor believes that the increase in these antibodies after an HIV-1 infection, slows down the progression of the infection towards development of AIDS and the high levels of IgM in newborns protect newborns from getting HIV-1 viremia from their infected mothers. Only 20 to 25% of babies, born of untreated mothers infected with HIV-1, are found to have the HIV-1 virus. Potential mechanisms for inhibiting HIV-1 infectivity of cells include, (but are not limited to): (i) “blocking” of HIV-1 viral entry through chemokine receptors either by steric hindrance or dimerization of chemokine receptors and (ii) inactivation of lymphocytes by binding to the CD3 receptor or other activating receptors e.g. chemokine receptors. IgM anti-leucocyte antibodies can cause dimerization of both chemokine receptors and the complex of TCR/CD3 receptors by binding and cross-linking these receptors as well as by binding to lipid rafts, which are associated with these receptors. Lipid rafts contain glycosphingolipids as well as phospholipids, which prior art has shown to be target antigens for IgM anti-lymphocyte auto antibodies. The binding of IgM anti-lymphocyte NAA to glycolipids and phospholipids has been described in Griggi et al, Scand. J of Immunol, 40: 77-82, 1994 and Stimmler et al, Archives of Internal Medicine 149: 1833-1835, 1989 and this material is incorporated herein by reference.

Chemokines, chemokine receptors, and other lymphocyte receptors (e.g. CD3 and other co-stimulatory molecules) are involved in inflammatory processes that involve leucocytes and endothelial cells. Examples of inflammatory processes include (but are not limited) auto-immune disorders (e.g. SLE, rheumatoid arthritis), asthma, atherogenesis, end-stage renal disease (ESRD) patients on hemodialysis, sarcoidosis, various viral, bacterial and parasitic infections, allograft and xenograft rejections, various forms of vasculitis, multiple sclerosis, interstitial lung and kidney inflammation and glomerulonephritis. While not being bound to a specific theory, the inventor believes that IgM anti-lymphocyte NAA through binding to chemokine receptors and other lymphocyte receptors could inhibit the above-mentioned inflammatory processes. Potential mechanisms for inhibition would include inhibition of chemokine receptor function after binding of IgM and more importantly inactivation of lymphocytes and/or macrophages after binding to chemokine receptors and non-chemokine receptors as for example, the CD3 receptor.

IgM anti-lymphocyte NAA also bind to endothelial cells and malignant cell lines. In this invention we show that IgM NAA, are polyreactive and hence, bind to the same or closely similar or different chemokine and non-chemokine receptors present on these cells. The inventor believes that some monoclonal IgM anti-chemokine receptor antibodies are polyreactive and bind to several different chemokine receptors as absorption of IgM with lymphocytes removes the IgM that binds to malignant cells, Neutrophils, eosinophils, macrophages, and endothelial cells even though these cells have different chemokine receptors and lack chemokine receptors present on lymphocytes.

It is believed that IgM by binding to chemokine and other non-chemokine receptors on endothelial and malignant cells inhibit the function of these cells. For example, there is prior art to show that chemokine receptors on malignant cells contribute to metastases of these cells. The inventor therefore believes that IgM, by binding to chemokine receptors on malignant cells and/or endothelial cells could, through steric hindrance or receptor dimerization, inhibit the growth and spread of malignant cells. Furthermore there is prior art to show that lymphocytes in lymph nodes and infiltrating leucocytes within the tumor mass secrete chemokines and other cytokines, all of which contribute to growth and metastases of tumor cells. The inventor therefore believes that IgM by binding to chemokine receptor and other “activation” receptors on leucocytes and malignant cells will, through this additional mechanism, also inhibit tumor growth and metastases. Finally, the inventor shows that malignant lymphoma cells (but not normal cells) have enhanced cell death at 37° C. when incubated with IgM. Hence, IgM through enhancing cell death of malignant cells could provide yet another mechanism for an anti-cancer effect.

Endothelial cells and leucocytes are also important in several inflammatory processes (e.g. allograft rejections, atherogenesis, vasculitis and inflammatory states of the brain). It is therefore believed that IgM anti-lymphocyte NAA by binding to chemokine receptors on leucocytes and endothelial cells could provide a protective role in inhibiting such inflammatory processes. Furthermore, IgM anti-lymphocyte NAA could also inhibit inflammatory processes by inhibiting receptors (e.g. CD3) that activate lymphocyte and macrophages.

The inventor believes that pooled IgM preparations contain a heterogenous group of antibodies that bind to chemokine or non-chemokine receptors on leucocytes, endothelial cells and malignant cells and that the binding of IgM to several of these receptors may add or have a synergistic effect in IgM mediated inhibition of viral infectivity, inflammatory states and malignant cell growth and spread.

### EXPERIMENTAL STUDIES

Methods/Procedures

Cell Lines

Sup T-1 is a lymphoma T cell line constitutively expressing the CXCR4 and CCR5 chemokine receptors. The Jurkat cell is a lymphoma T cell line constitutively expressing the CXCR4, but not the CCR5 receptor. These cell lines are obtained from the AIDS Reagent Program at NIH.

An HOS osteosarcoma cell line is co-transfected with CD4 and either CXCR4 or CCR5 genes to produce HOS-CD4, HOS-CD4-CXCR4 and HOS-CD4-CCR5 cells. Ghost CCR5 and Ghost CXCR4 are HOS-CD4 cells co-transfected with the HIV-2 LTR driving hGFP construct and either CCR5 or CXCR4 genes, respectively. The cell line and the transfectants are obtained from the AIDS Reagent Program at NIH.

A glioblastoma cell line, U373-MAGI, is co-transfected with CD4 and either CXCR4 or CCR5 to produce U373-MAGI-CXCR4 and U373-MAGI-CCR5, respectively. Again, the cell line and the transfectants are obtained from the AIDS Reagent Program at NIH.

All of the transfected cell lines stably express CCR5 or CXCR4, with the U373-MAGI cells having the highest expression of these receptors.

Human peripheral blood lymphocytes (“PBL”) is activated with IL-2 to enhance CCR5 expression. PBL (2×10⁶ cells in 1 ml RPMI culture media containing 10% fetal calf serum are activated by initially pre-treating ficol/hypaque separated PBL with IL-2 (40 units/ml) and phytohemagglutinin (“PHA-P”, 5 mcg/ml) and then washing the PBL after the cells are cultured at 37° C. in about 5% CO₂ for 24 to 48 hours. Such PHA pre-treated cells are then kept growing for about another 6 to 7 days supplemented with 20% fetal calf serum and IL-2 (40 units/ml) before being used in chemokine binding assays.

HIV-1 Viruses

The R5 HIV-1 viruses (8397, 8442, and 8658) used to infect Ghost CCR5 is obtained from Dr. Homayoon Garadegan at Johns Hopkins University. The X4 virus IIIB and RF used to infect Ghost CXCR4 is obtained from the AIDS Reagent Program at NIH.

IgM Preparations and Sera

Studies were performed with IgM that was purified from heat-inactivated sera (56° C.) of normal individuals or from patients. IgM was prepared from sera with Sephacryl S-300 HR size exclusion column chromatography. IgM was not precipitated from sera with hypotonic dialysis as this process reduced the biological activity. Any contaminating IgG was removed from the IgM preparation by re-passage of purified IgM through a Sephacryl/S-300 HR column and by exposure to Agarose-protein G and Agarose bound to goat anti-human IgG (Sigma). We employed these procedures basically to remove low molecular weight substances (e.g. chemokines, anti-viral drugs) and IgG anti-HIV-1 antibodies that could affect our data. Serum protein electrophoresis and immunoelectrophoresis revealed that these IgM preparations, obtained by size exclusion chromatography, contained IgG (<1%), albumin (<3%), and other proteins (<1%). We did not want to affinity purify these antibodies as such procedures, e.g. binding of IgM to mannan binding protein or binding of IgM to agarose coupled with goat anti-human IgM yielded 10-15% of the starting IgM and has the potential of depleting certain IgM subsets. Instead, in several experiments we used IgG, IgA, albumin and alpha 2 macroglobulins to determine if our observations could be explained by some of these minute contaminants. No detectable RANTES and SDF-1α was present in these IgM preparations when analyzed by ELISA and Western blot techniques.

We obtained sera from normal uninfected healthy individuals, asymptomatic patients with HIV-1 infection, patients diagnosed as AIDS and patients with end stage renal disease (ESRD) on hemodialysis. Sera were defined as “HIV IgM” if the HIV-1 infected patient was asymptomatic, on no antiviral agents and with >500 CD4 positive cells per ml and with a RNA viral load of <2,000 copies. Sera were defined as “AIDS IgM” if patients had an opportunistic infection and had <150 CD4 positive cells per ml and an RNA viral load of >10,000 copies with or without antiviral agents. To obtain a sufficient quantity, IgM from nine AIDS patients was pooled, as was IgM from seven HIV patients. IgM from seven ESRD patients was also pooled. Data presented in figures are from pooled IgM unless otherwise indicated.

The culture supernatants of EBV transformed human B cell clones are separated by Sephacryl S-300 HR column chromatography, which separates proteins by size. The human B cell clones are derived from B lymphocytes isolated from the blood of a patient with SLE. The B cell clones are developed by infecting B cells with the EBV virus, which makes the B cells immortal and capable of secreting a specific antibody, i.e., IgM. More particularly, non-T cells are isolated from PBL after removal of T cells using a sheep erythrocyte resetting technique. About 2×10³ non-T cells in about 0.1 ml RPMI 1640 cell culture media containing about 10% fetal calf serum are added to each well of a 96 well plate. To each well is then added about 50 lambda of EBV-containing B95-8 cell line supernatant. Before incubation, about 10⁴ allogenic irradiated (3,000 rads) PBL in 0.05 ml are added as feeder cells. The plates are incubated at 37° C. in about 5% CO₂. The culture medium is replaced about every 4 to 5 days. After about 3 to 4 weeks, B cell lines appear as “clumps” in the wells. Feeder cells die during this period. When the “clumps” appear, these clumped cells are transferred to a 24-well plate, i.e., cells from one well are transferred into a single larger well. Culture media is changed when the media changes to a yellowish color, usually about 3 to 5 days. After about 2 weeks, supernatants are checked for IgM antibody. Wells containing lines with desired antibody specificity are further subcloned with limiting dilution in a 96-well plate. About 10⁵ feeder cells are added to each well containing these lines. Supernatants are rechecked to isolate clones with desired antibody specificity. Supernatants are refrigerated, but not frozen as IgM can precipitate out. Clones secreting IgM antibodies that are useful in inhibiting HIV-1 infectivity are cryopreserved. Supernatants from such clones usually contain about 0.5 to about 0.7 μg/ml antibody. Clones of particular interest can be fused with K6H6/B5 plasmacytoma cell line to develop hybridomas. The clones are screened to identify and obtain those clones that react with CCR5 and CXCR4 chemokine receptors present on the transfected cells. Such clones have increased IgM binding by flow cytometry to the HOS-CD4 transfectants (i.e., HOS-CD4-CXCR4 and HOS-CD4-CCR5) when compared to the HOS-CD4 control. Two clines, CK12 and CK15 secreting IgM with increased binding to HOS-CD4 transfectants were identified in this manner.

Any contaminating IgG is removed from the IgM preparations that are isolated from the sera and the culture supernatants by exposure to both protein G-Agarose (available from Sigma) and goat anti-human IgG (Fe specific)-Agarose (available from Sigma).

IgM is also obtained using Sephacryl S-300 HR column chromoatography from sera of a patient diagnosed with Waldenstrom macroglobulinemia (a form of B cell lymphoma) and which, on serum protein electrophoresis, has a single peak for IgM (monoclonal). This latter IgM preparation is hereinafter referred to as “Waldenstrom IgM.” Affinity purified pooled normal IgM was also obtained from Accurate chemicals.

Chemokines

Four chemokines preparations are used in the following studies. RANTES, SDF-1α and biotin-labeled SDF-1α and RANTES are obtained from Becton Dickinson of La Jolla, Calif. Radio-labeled RANTES (referred to as “I¹²⁵ RANTES” or “I²⁵¹”) is obtained from NEN Life Science of Boston, Mass. RANTES binds to CCR5, while SDF-1α binds to CXCR4.

IgG Antibodies

Clones 2D7 and CTC-5 are murine IgG monoclonal antibodies specific for CCR5 when expressed on intact cells. Clone CTC-5 in addition binds to linearized CCR5 in Western blots (R&D Systems, Minneapolis, Minn.). Clones 12G5 (IgG 2a) is a murine IgG monoclonal that bind to CXCR4 receptors on intact cells (R&D Systems). Clone 4G10, a murine IgG monoclonal that binds to the N-terminal region of CXCR4 was a kind gift from Dr. Chris Broder. Clones W6/32 and L243 are murine IgG monoclonals that bind to non-polymorphic determinants shared by HLA-A, B and HLA-DR, respectively (ATCC at NIH). Leu 3a is an IgG murine monoclonal reactive to CD4 (BD-Pharmigen, San Diego, Calif.). OKT3 is an IgG murine monoclonal antibody reactive to CD3 present on T lymphocytes.

Quantitation of IgM Binding to Cells

Flow cytometry is used to quantify IgM binding to the cells. Prior to flow cytometry, about 1×10⁵ cells are initially incubated at about 4° C. with about 150 nM of each of Normal IgM, HIV IgM and AIDS IgM. The cells are then washed, followed by staining with fluorescein-isothiocyanate (“FITC”) goat anti-human IgM (Fe specific). Binding of IgM to human peripheral blood T lymphocytes is quantified by two color flow cytometry, i.e., using phycoerytherin (“PE”)-labeled anti-CD3 and FITC-labeled goat anti-human IgM (Fe specific).

IgM Inhibition of I¹²⁵ RANTES Binding to CCR5

Various studies are employed to determine if IgM inhibits binding of chemokines to chemokine receptors. In this study, the focus is whether IgM with anti-CCR5 activity can inhibit binding of I¹²⁵ RANTES to CCR5. This study is performed with affinity purified IgM and with supernatants from EBV transformed B cell clones, specifically, CK15. Controls are supernatants containing IgM Rheumatoid factor and purified human IgG.

The first approach is to determine if affinity purified Accurate IgM and/or CK15 IgM inhibit binding of I¹²⁵ RANTES to non-denatured, crude membrane proteins obtained from U373-MAGI-CCR5. Here, each of Accurate normal IgM and CK15 IgM or 500 fold unlabeled RANTES relative to labeled RANTES in varying molar concentrations ranging from about 10⁻⁶ to about 10⁻¹ are incubated with about 5 μg of non-denatured U373-MAGI-CCR5 membrane proteins for about 1 hour at room temperature in the presence of Ca⁺² and Mg⁺² and a protease inhibitor. At the end of the incubation, about 0.25 μM I¹²⁵ RANTES available from NEN Life Science is added to each mixture, and each mixture is further incubated at room temperature for about another 2 hours. Each mixture is then harvested over fiberglass filters and washed three times to remove unbound I¹²⁵ RANTES. Specific I¹²⁵ RANTES binding is calculated by subtracting the counts of radioactivity per minute (“c.p.m.”) of I¹²⁵ RANTES when used with 500 fold molar excess of unlabeled RANTES from the data obtained with I¹²⁵ RANTES.

In a second approach, IgM inhibition of I¹²⁵ RANTES binding to CCR5 is detected by Western blotting. Here, about 250 μg of non-denatured U373-MAGI-CCR5 membrane proteins are incubated, under non-reducing conditions, with each of about 0.1 nM Normal IgM, 350 nM IgG, 0.4 mcM unlabeled RANTES and a culture media control at room temperature for about 1 hour prior to adding about 1.0 nM I¹²⁵ RANTES available from NEN Life Science to each mixture. After about 2 hours incubation, about 5 nM of a cross-linker (BS-3 available from Pierce of Rockford, Ill.) is added to each mixture to crosslink amine residues on I¹²⁵ RANTES bound to CCR5. Also, I¹²⁵ RANTES in the absence of U373-MAGI-CCR5 membrane proteins is used. Each mixture is then electrophoresed onto 12% gel SDS-PAGE, and radiographs of the gel are obtained.

IgM Inhibition of RANTES Binding to CCR5 on Intact Cells

A procedure is used to determine if IgM inhibits binding of RANTES to CCR5 receptors present on intact cells, e.g., U373-MAGI-CCR5E and IL-2-activated human lymphocytes. About 1×10⁵ cells are initially incubated at room temperature for about 45 minutes with RPMI media containing about 10% fetal calf serum or with about 150 nM of affinity purified Accurate IgM or with about 400 nM purified human IgG or with about 5 nM, about 1.25 nM or about 0.45 nM of CK15 IgM prior to adding about 1 microgram of RANTES to each mixture. The cells are then re-incubated for about 90 minutes at 40 C and then washed at 4° C. Goat anti-RANTES antibody (obtained from R&D of Minneapolis, Minn.) is then added, and the cells are incubated for about 45 minutes at 4° C. prior to being washed and then stained with FITC rabbit anti-goat antibody. The quantity of FITC-labeled RANTES binding to CCR5 on these cells is analyzed by flow cytometry.

IgM Inhibition of SDF-1α Binding to CXCR4 on Intact Cells

A procedure is used to determine if IgM inhibits binding of SDF-1α to chemokine receptors (i.e., CXCR4) present on intact Sup T-1 cells. About 1×10⁵ Sup T-1 cells are initially incubated at room temperature for about 45 minutes with RPM1 media containing about 10% fetal calf serum or with about 150 nM of each of Normal IgM, HIV IgM and AIDS IgM or with about 5 nM of each of Waldenstrom IgM and CK15 IgM prior to adding about 25 ng of biotin-labeled SDF-1α to each mixture. The cells are then re-incubated for about 90 minutes at 4° C. Following re-incubation, FITC avidin is added to the cells, and the cells are washed. The quantity of FITC-labeled SDF-1α binding to CXCR4 is analyzed by flow cytometry.

IgM Inhibition of Radio-labeled IL-2 Binding to Human Lymphocytes

Additional procedures are performed to determine if IgM inhibition of chemokines binding to CCR5 or CXCR4 is indeed specific for chemokines. More particularly, these procedures are used to determine if IgM, through some non-specific mechanism, also inhibits binding of radio-labeled IL-2 to the IL-2R present on phytohemagglutinin-activated PBL using, methods as previously described in, for example, Teshigawara, K. et al., J. Exp. Med., 165:223-238 (1987), which is incorporated herein by reference. Specifically, three day phytohemagglutinin-activated PBL (1×10⁶) is incubated with I¹²⁵ labeled IL-2 (available from NEN Life Science), and the I¹²⁵ labeled IL-2 bound to PBL is quantified by overlaying the PBL over oil and centrifuging the microfuge tube to separate unbound I¹²⁵ labeled IL-2 from the cell pellet. Radioactivity of I¹²⁵ is quantitated in the cell pellet. In these procedures, PBL is interacted with each of excess unlabeled IL-2 (2.0 mcM), pooled human IgG (300 nM), IgM Rheumatoid factor and affinity purified Accurate IgM (100 nM) prior to adding I¹²⁵ labeled IL-2.

Immunoprecipitation Technique to Detect Normal IgM Binding to Solubilized Cell Membrane Receptors:

This procedure was performed to determine if normal human IgM will form complexes with receptors solubilized from membranes of cells. Agarose beads containing covalently bound goat anti-human IgM is used to immunoprecipitate IgM complexed to the receptors. The receptor in the complex is then identified with monoclonal antibodies specific for the receptor.

Jurkat or U937 cells (80×10⁶) were incubated for 30 min at 4° C. with 10 ml of 100 mM (NH4)₂SO₄, 20 mM Tris HCl (pH 7.5) containing 10% glycerol, 1% Cymal −5 (Anatrace, Maumee, Ohio) and 1 tab mini-complete (Roche) to solubilize membrane receptors with minimal denaturation. IgM/receptor complexes were formed by interacting 40 μl of cell lysate with 40 μg of normal IgM that was pre-absorbed with goat and mouse IgG to remove any IgM having reactivity to goat or mouse IgG. The mixture of IgM/cell lysate was then interacted with 30 μl of a 20% Agarose beads that were covalently bound to goat IgG anti-human IgM (Sigma) to precipitate out normal IgM/receptor complexes in the cell lysate. The agarose bead with bound IgM/receptor complexes was washed ×2 (700 rpm) with buffer and then interacted with monoclonal mouse IgG specific for the receptor. Bead complexes were re-washed and then incubated with FITC goat anti-mouse IgG (Fc specific). After re-washing, beads were fixed with 2% paraformaldehyde and then flow cytometry was used to detect receptor bound to normal IgM. Human albumin was added to the bead complexes prior to adding mouse IgG anti-receptor antibody to prevent non-specific binding of mouse IgG to the agarose beads. As controls, the same experimental procedure was followed, except immune complex formation was prevented in the initial step by adding media (instead of cell lysate) to normal IgM or adding lysate with no IgM. We introduced the flow cytometric technique to circumvent the problems inherent in using Western blots to detect immunoprecipitated chemokine receptor complexed to IgM. The Western blot technique linearlizes and changes chemokine receptor conformation and hence detection of chemokine receptors becomes problematic as most of the murine IgG monoclonals bind to conformational dependent epitopes on the chemokine receptor.

Chemotaxis Assay

A chemotaxis assay is performed with each of Normal IgM, HIV IgM, AIDS IgM and Waldenstrom IgM at concentrations of about 20 nM, about 40 nM, about 100 nM and about 200 nM IgM using 24-well Costar transwell tissue culture inserts with 5 micron polycarbonate filters. For each assay, the IgM is placed in the upper transwell containing about 2×10⁴ Jurkat cells in about 0.1 ml RPM1 containing about 2% human albumin. About 30 minutes later, approximately 50 ng of SDF-1α is added to the bottom well containing about 0.6 ml of the same media as in the upper well. After about 4 hours, cells migrating to the bottom well are enumerated by flow cytometry. The chemotaxic index (“CI”) is calculated by dividing the total number of cells migrating in the presence of SDF-1α by the number of cells migrating in the absence of SDF-1α. The baseline chemotactic index of SDF-1α alone (i.e., without IgM) is about 3.1.

Measurement of Intracytosolic Ca⁺² Flux

Assays are performed to determine intracytosolic Ca⁺² flux using known methods, for example, as described in Haverstick, G., MD, Molecular Biol. of Cell. 4:173-184 (1993), which is incorporated herein by reference. In one assay, about 45 nM of HIV IgM is added to Jurkat cells at a time of about 20 seconds. Approximately 60 seconds later, about 100 ng of SDF-1α is added, and the magnitude of change in cytosolic Ca⁺² after adding SDF-1α is measured. A second assay is done using about 45 nM of AIDS IgM in place of HIV IgM. In a third assay, no IgM is added, but SDF-1α is still added at a time of about 80 seconds.

Temperature Dependence for the Cytolytic Effects of IgM Anti-Leukocyte Antibody

Temperature dependence for the cytolytic effects of IgM anti-leukocyte antibody is evaluated by a complement dependent microlymphocytotoxicity assay. Various dilutions of IgM antibody are reacted for 1 hour with either 2×10⁵ PBL or IL-2-activated PBL (7 days) before adding fresh rabbit serum as a source of complement. After about 2 hours, the cells are washed twice before adding trypan blue and enumerating dead cells that stain blue. Experiments are performed at 15° C. and 37° C.

IgM Inhibition of HIV-1 Infection of Cells

It has been observed that the HIV-1 R5 virus utilizes CCR5 receptors for cell entry, while the HIV-1×4 virus uses CXCR4 receptors. Studies are conducted, therefore, to determine whether IgM inhibits HIV-1 infectivity in light of such observations.

In these studies, Ghost CCR5 and Ghost CXCR4 transfectant cell lines are infected with HIV-1. The Ghost cells are derived from HOS cells transfected with either CCR5 or CXCR4 genes and also co-transfected with the HIV-2 LTR driving hGFP construct. The hGFP construct enables cells infected with HIV-1 virus to emit a green fluorescence so that the number of infected cells can be quantified using flow cytometry. These cell lines are particularly suited for these studies because single-cycle viral replication can be detected in less than 48 hours.

About 2×10⁴ each of Ghost CCR5 and CXCR4 cells are separately cultured for about 12 hours in about 1 ml RPM1 media containing about 10% fetal calf serum in a 12-well plate. Normal IgM is then added to each of the Ghost CCR5 and CXCR4 cells about 30 minutes prior to adding the R5 HIV-1 virus to Ghost CCR5 and the X4 HIV-1 virus to Ghost CXCR4. Both virus and antibody are present throughout the 48-hour culture period. No polybrene is used to enhance viral entry into the cells. The same procedure is repeated twice, replacing Normal IgM first with HIV IgM and then AIDS IgM.

After the 48-hour incubation period, cells are harvested and fixed in formalin. Infected cells emitting green fluorescence are enumerated with flow cytometry.

Additionally, similar data is obtained when the virus or IgM antibody is washed about 4 hours after incubating with Ghost cells.

HIV-1 infection of Human PBL/SCID mice: We employed (with modifications) the procedure developed by Mosier and as described in Torbett et al, Immunol Reviews 124: 139-164, 1991, which is incorporated herein by reference. Seven to eight week old female CB17 SCID mice, purchased from Harlan Sprague Dawley, Indianapolis, Ind. and having <1 μg per ml of mouse IgM in their plasma were injected intraperitoneally with freshly isolated 25-35×10⁶ PBL in 1 ml RPMI containing 10% FCS and antibiotics (RPMI culture media). Two hours later mice were re-injected intraperitoneally with 10⁵ TCID₅₀ HIV-1 virus in 1 ml RPMI culture media. One ml of IgM at 1 mg/ml, obtained from the same PBL donor, was injected intraperitoneally, either immediately after the HIV-1 injection or 48 hours later. The same dose of IgM was injected every five days until day of sacrifice as kinetic studies revealed that human IgM in mouse plasma attained peak levels of 40-50 μg by day two and 8-10 μg per ml by day five after the intraperitoneal dose. Mice were sacrificed three weeks after the human PBL injection. Percent human CD3 and CD4 positive T lymphocytes in spleen cells were quantitated with FITC labeled mouse anti-human CD3 or CD4 (BD Pharmigen) using flow cytometric techniques. Secondly, spleen cells were co-cultured with five to seven day IL2-activated autologous PBL to quantitate HIV-1 in spleen cells. In co-culture studies 2×10⁶ spleen leucocytes in 1 ml RPMI culture media were co-cultured with 2×10⁶ IL-2 activated (5-7 days old) human PBL in 1 ml RPMI culture media containing human IL2 (30 units/ml). Co-cultures were fed at weekly intervals with five-day-old 2×10⁶ IL2-activated autologous PBL. p24 antigen in co-culture supernatants was quantitated after three weeks of co-culture using an ELISA kit. With this protocol (i.e. single dose of virus and sacrifice at three weeks) one could not detect viremia after the first week. Studies on SCID mice were approved by our Institutions Animal Care and Use Committee.

### Results

The results of the various experimental studies indicate that IgM autoantibodies bind to chemokine receptors and the CD3 antigen present on lymphocytes and other cells.

Binding of IgM to Cells and Cell Membrane Proteins

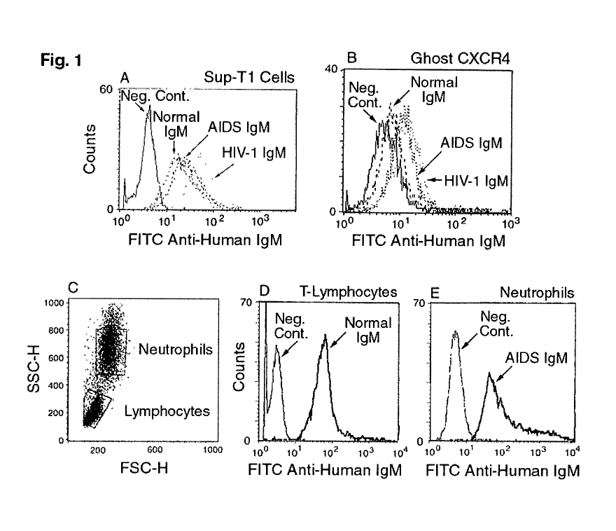

The data from the above-discussed studies shows that IgM autoantibodies bind to receptors present on normal lymphocytes and malignant cells. As seen in FIGS. 1A and 1B, Normal IgM, AIDS IgM and HIV IgM contain IgM antibodies that bind to Sup T-1 (FIG. 1A) and Ghost CD4-CXCR4 cells (FIG. 1B). As seen in FIGS. 1D and 1E, Normal and AIDS IgM contains antibodies that bind to T-lymphocytes isolated from peripheral blood (FIG. 1D) and neutrophils isolated from peripheral blood (FIG. 1E). The negative control in each figure indicates that no IgM was incubated with the various cells.

Binding of IgM to Solubilized Membrane Receptors:

We employed this approach to more definitively demonstrate that pooled normal and AIDS IgM binds in a specific manner to membrane receptors. Non-linearized receptors solubilized from cell membranes was allowed to form complexes with normal human IgM. The receptor in the IgM complex is identified by initially immunoprecipitating the IgM complex with agarose beads, covalently, bound with goat anti-human IgM. The receptor in the immunoprecipitated complex was then identified with receptor specific monoclonal antibodies and employing flow cytometry.

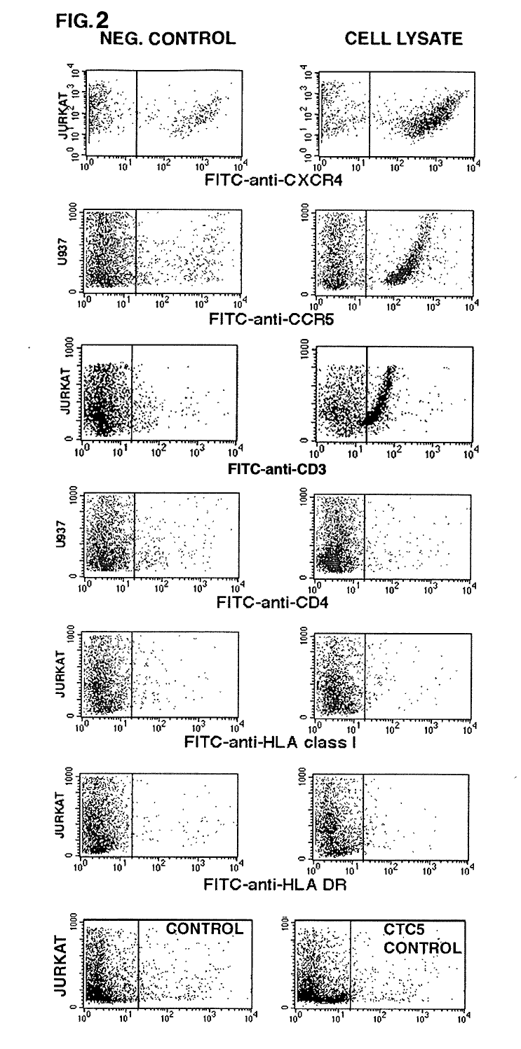

As noted in FIG. 2, normal IgM formed complexes with CXCR4, CCR5 and CD3 but not CD4 receptors present in the lysates. Similarly IgM did not form complexes with HLA-Class I and HLA-DR receptors. Controls using the same procedure, but with either no IgM or no lysate to form the complex, revealed no detectable receptor that non-specifically bound to the agarose beads (see left panels of FIG. 2). In another control, IgM/receptor complexes from Jurkat cells (lacking CCR5) were examined with CTC-5 anti-CCR5 monoclonal. The CTC-5 antibody did not bind to any of the receptors in the IgM immunoprecipitate, indicating that monoclonal antibody specificity is retained with the flow cytometric detection system (see very bottom panels of FIG. 2). Furthermore, lack of HLA-Class I, HLA-DR and CD4 receptor detection in the IgM complexes cannot be attributed to lack of these receptors in the lysate as the same murine IgG monoclonal used for detecting receptors in the complex clearly detected a high density of these receptors on intact Jurkat and U937 cell membranes using flowcytometry. One can make the following conclusions based on the results of the immunoprecipitation studies. First, the cell receptors detected in this assay system bound to IgM especially since we used agarose beads covalently bound to goat anti-human IgM to capture IgM containing complexes. Secondly, these studies clearly show that IgM binding to chemokine receptors has specificity as IgM failed to form complexes with other carbohydrate containing receptors, e.g. CD4, HLA-Class I and HLA-DR receptors. Similar data were obtained with AIDS IgM and IgM secreted by clones CK15 and CK12. There was no quantitative difference noted comparing receptor binding between normal and AIDS IgM.

IgM Inhibition of Chemokine Binding to Receptors

IgM autoantibodies inhibit binding of radio-labeled RANTES to CCR5 receptors but not binding of radio-labeled IL-2 to IL-2 receptors (i.e., IL-2R). This supports the concept that IgM-mediated inhibition is indeed specific for chemokines. Moreover, normal IgG does not inhibit radio-labeled RANTES from binding to CCR5 receptors.

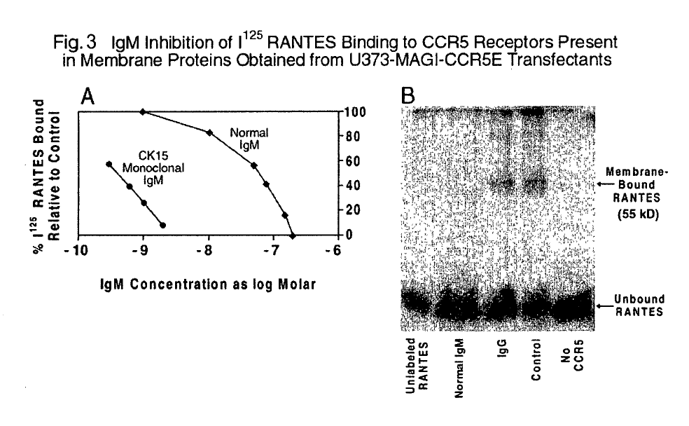

Representative data from initial studies conducted to determine if affinity purified Accurate normal IgM and/or CK15 IgM inhibit binding of I¹²⁵ RANTES to non-denatured, crude membrane proteins obtained from U373-MAGI-CCR5E is depicted in FIG. 3A. As seen therein, accurate normal IgM and the CK15 IgM inhibit binding of I¹²⁵ RANTES to CCR5 in a dose-dependent manner. Pooled Normal IgG and IgM Rheumatoid factor, even when used at 10-6 M, fail to inhibit binding of I¹²⁵ RANTES to CCR5. Unlabeled RANTES inhibits I¹²⁵ RANTES binding in a dose-dependent manner with a kD of 0.095 nM.

IgM inhibition of the binding of I¹²⁵ RANTES to CCR5 as detected by Western blotting is depicted in FIG. 3B. As seen therein, affinity purified Accurate IgM and unlabeled RANTES inhibit binding of I¹²⁵ RANTES to CCR5. Neither the pooled human IgG nor the RANTES protein control inhibits binding of I¹²⁵ RANTES to CCR5. This latter observation would appear to indicate that IgM inhibition of I¹²⁵ RANTES binding to CCR5 is a result of receptor blockade and is specific for IgM having a specificity for CCR5.

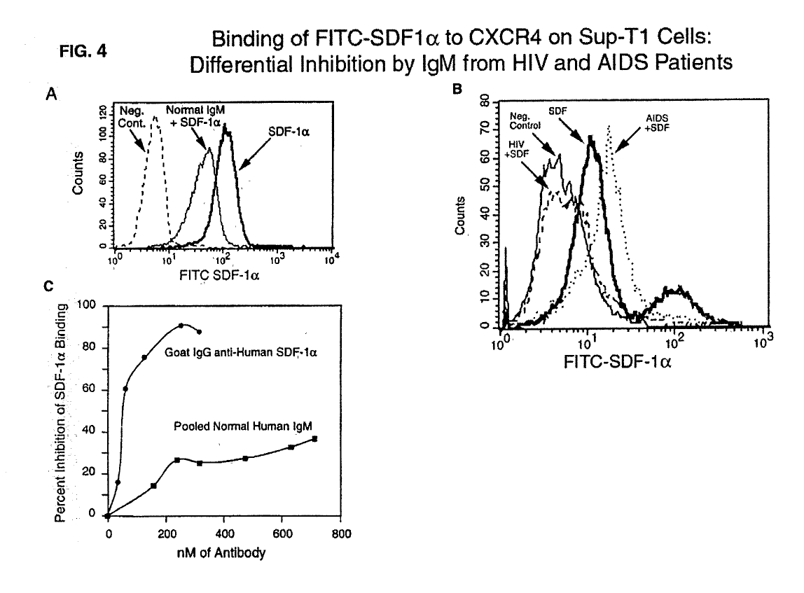

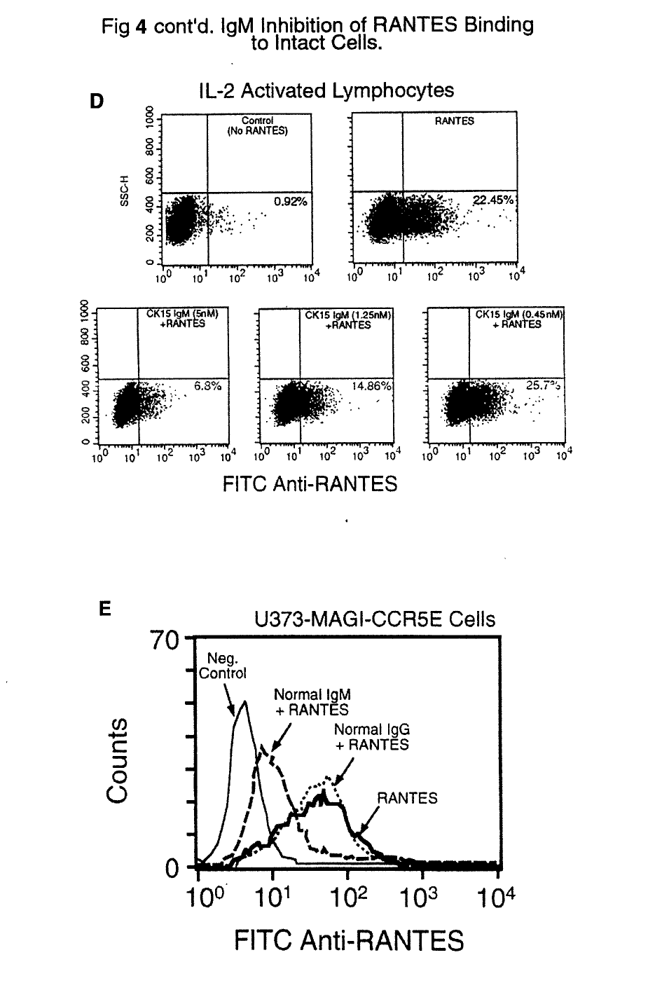

Additional studies were performed using Sup T-1 cells and IL-2-activated lymphocytes to determine if IgM inhibits chemokines from binding to their receptors present on intact cells. As seen in FIG. 4A, normal IgM partially suppresses biotin SDF-1α binding to intact Sup T-1 cells by about 30 to 40%. As seen in FIG. 4B, HIV-1 IgM completely suppresses the binding of SDF-1 to intact Sup T-1 cells while AIDS IgM does not. A negative control is used to indicate background fluorescence of cells without IgM and SDF-1α. “SDF” indicates SDF-1α binding in the absence of IgM. Note that a small subset (15%) of Sup T-1 cells had much stronger binding to biotin SDF-1α. As seen in FIG. 4C, increasing the dose of normal IgM fails to increase the inhibitory effect of normal pooled IgM on SDF-1 binding to Sup T-1 cells. As indicated in FIG. 4D, about 22.45% of IL-2-activated lymphocytes bound to RANTES. In the presence of about 5 nM CK15 IgM, the binding decreased, with about 6.8% of the lymphocytes binding to RANTES. Less inhibition is observed with less CK15 IgM. As indicated in FIG. 4E, affinity purified Accurate IgM, but not human IgG, inhibited binding of RANTES to intact U373-MAGI-CCR5E cells.

An additional study to determine if IgM inhibition of chemokines binding to their receptors is indeed specific for chemokines shows that binding of I¹²⁵-labeled IL-2 is inhibited by unlabeled IL-2 (>90%) and anti-TAC (anti-IL2R murine IgG antibody) but not by pooled human Accurate IgM, even when used at 100 nM. The results are shown in Table 1 below.

TABLE 1
INHIBITION OF I¹²⁵-LABELED IL-2
BINDING TO IL-2R ON ACTIVATED PBL

| | |
|---|---|
| Control Media | 9,468 |
| Human IgG | 8,293 |
| Human IgM | 8,809 |
| Murine anti-TAC | 1,948 |
| Unlabeled IL-2 | 2,353 |

The above results, therefore, indicate that inhibition of SDF-1α binding to CXCR4 by Normal IgM and HIV-1 IgM is specific. Data demonstrating inhibition of biotin SDF-1α binding to Sup T-1 cells when using Normal IgM and HIV IgM cannot be explained on basis of stearic hindrance as AIDS IgM has similar binding to Sup T-1 cells and to solubilized CXCR4 receptor and yet fails to inhibit SDF-1α binding (see FIG. 1A). CK15 IgM, which inhibited binding of RANTES to CCR5 failed to inhibit binding of SDF-1α to CKCR4 indicating that inhibition of chemokines to their receptors is indeed specific. While not wishing to be bound to any particular theory, one possible explanation is that IgM anti-leukocyte autoantibodies are heterogeneous and recognize different epitopes on the chemokine receptor. It is, therefore, possible that AIDS IgM lacks the subset of IgM antibodies that inhibit SDF-1α binding, even though the AIDS IgM binds to intact chemokine receptors as will be evident from the data obtained in the chemotaxis assay (described in detail below).

Effect of IgM on Chemotaxis and Intracytosolic Ca⁺²

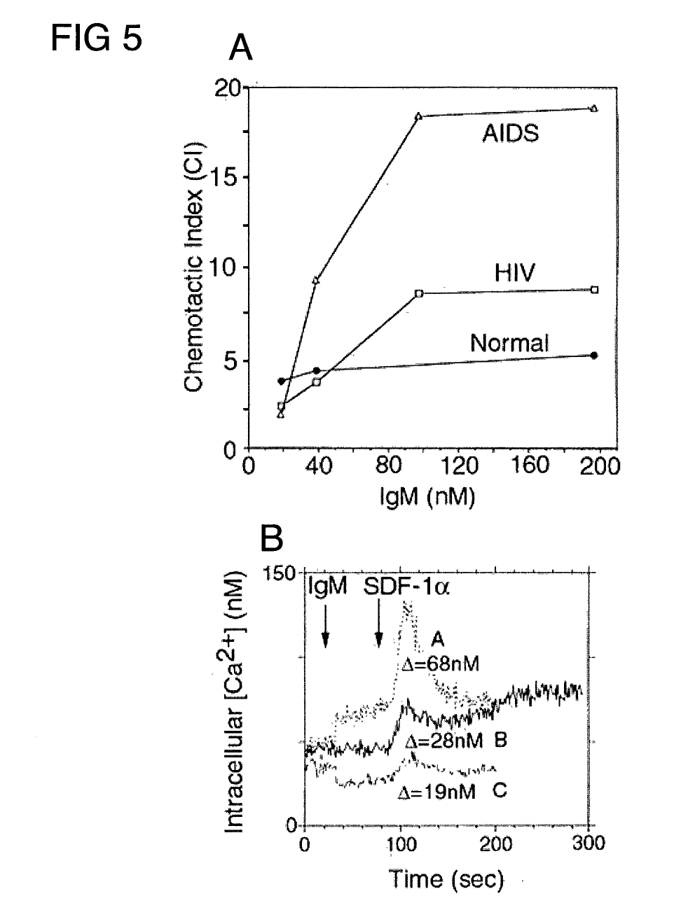

The possibility of heterogeneity of IgM is analyzed in functional assays of SDF-1α induced chemotaxis (FIGS. 5A) and intracellular Ca⁺² flux (FIG. 5B).

All IgM preparations in the absence of SDF-1α did not affect baseline chemotaxis. In the presence of SDF-1α, however, pre-treatment of Jurkat cells with the various IgM preparations affected chemotaxis (see FIG. 5A). Particularly, as seen in FIG. 5A, all pooled IgM preparations enhanced chemotaxis, with AIDS IgM showing the most enhancement. Enhanced migration into the bottom well after adding IgM to the upper well was mediated by chemotaxis-induced migration and not from some non-specific process, as adding 500 ng of SDF-1α to the upper transwell totally inhibited this enhanced migration. Control CI in the presence of SDF-1α is about 2.8. Waldenstrom IgM inhibited chemotaxis (CI=about 1.1). CK12 IgM and CK15 IgM mildly enhanced chemotaxis with the CI varying from about 5.3 to about 7.1, which is significantly more than that observed with SDF-1α alone (CI=3.1).

These findings prompted a determination of whether IgM similarly affected changes in cytosolic Ca⁺² that occur when chemokines bind to the CXCR4 receptor present on Jurkat cells. Representative data from three such experiments is depicted in FIG. 5B, where the symbol Δ indicates the magnitude of change in cytosolic Ca⁺² after adding SDF-1α. Tracing A represents Jurkat cells with AIDS IgM; tracing C represents Jurkat cells with HIV IgM; and tracing B represents Jurkat cells with no IgM. As seen in FIG. 5B, none of the IgM antibodies in the absence of SDF-1α (i.e., prior to adding of SDF-1α) elicit a rise in intracellular Ca Tracing A indicates that AIDS IgM enhances the rise in the intracellular Ca⁺² response to SDF-1α. Tracing C indicates that no enhancement occurs with HIV IgM.

Specificity of IgM interaction with CXCR4 in these Ca⁺² flux assays is ascertained by performing similar studies on another receptor present on Jurkat cells, i.e., the CD3 receptor. No enhancement in cytosolic Ca⁺² is observed by adding AIDS IgM prior to stimulating CD3 with OKT3, a murine IgG anti-CD3 antibody. Such data provides more evidence to support specificity of IgM binding to the CXCR4 receptor. Furthermore, these findings clearly support the concept of functional heterogeneity within IgM anti-lymphocyte autoantibodies, with AIDS IgM containing IgM that predominantly enhances chemotaxis and Ca⁺² flux after binding to the chemokine receptor. Conversely, AIDS IgM could lack IgM that inhibits chemotaxis and Ca⁺² flux.

Effect of IgM on Activation and Proliferation of Human Lymphocytes

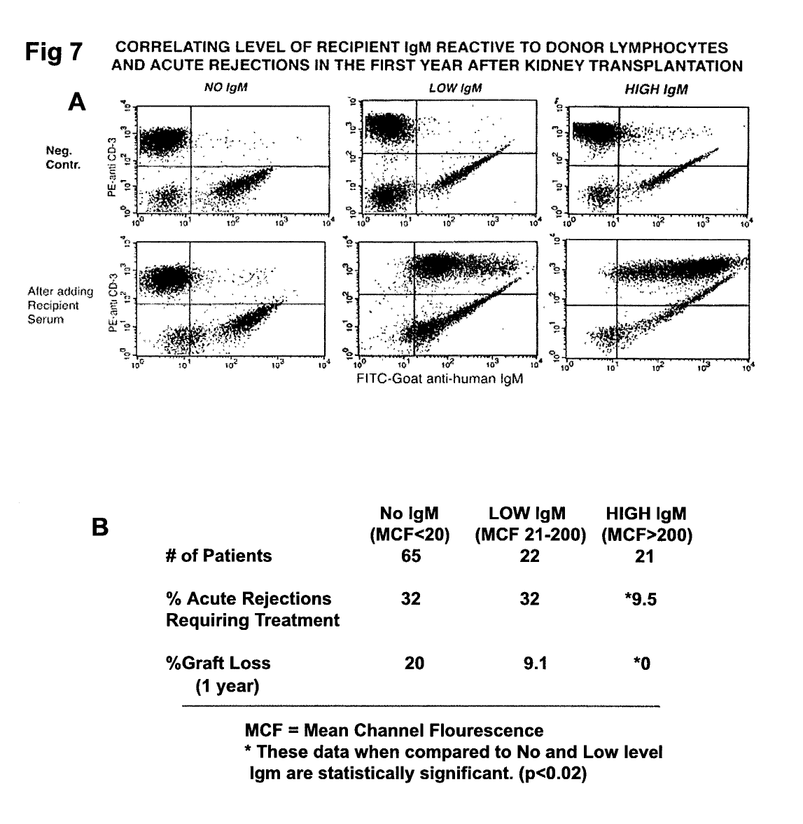

The finding that normal human IgM binds to the CD3 receptor in the immunoprecipitation experiments (see FIG. 2) and the observation (see FIG. 7) showing that kidney transplant recipients (ESRD) with high IgM anti-lymphocyte antibodies had no or minimal rejections prompted us to determine if IgM anti-lymphocyte antibodies had an inhibitory effect on lymphocyte activation and proliferation. We evaluated the effect of pooled ESRD IgM in a mixed lymphocyte culture (MLC) assay. In this assay 0.5×10 ⁶ peripheral blood monoculear cells (PBL) isolated from individual A are co-cultured in quadruplicate with 0.5×10⁶ mononuclear cells from a non-HLA identical individual B in 0.5 ml RPMI culture media, containing 10% fetal calf serum. Different quantities of pooled ESRD IgM are added to the co-cultures prior to initiation of the culture. At the end of five days in culture at 37° C. in 5% CO₂, cells are exposed to ³[H] Thymidine and 24 hours later cells are harvested on a filter paper matrix and washed. The extent of lymphocyte proliferation is quantitated by amount of ³[H] Thymidine uptake into proliferating cells using liquid scintillation. Representative data from two of five experiments are presented in Table 2.

In the MLC assay, T lymphocytes from either individual get activated in response to differences between HLA-class II antigens present on macrophages and B lymphocytes in the two individuals. Activated T cells subsequently proliferate. As seen in Table 2, pooled ESRD IgM significantly inhibited activation and proliferation of T cells in these co-cultures.

The inventor believes that this inhibitory effect is mediated by IgM binding to the CD3 receptor thus preventing T lymphocytes from effectively recognizing the allogeneic HLA-class II antigens. The CD3/TCR is the primary recognition receptor on T cells. Failure of T cells to recognize allogeneic HLA-DR cannot be attributed to IgM “masking” of HLA-DR as IgM does not bind to HLA-DR antigens (see FIG. 2). Such a conclusion is plausible as there is prior art to show that murine IgG anti-human CD3 monoclonal antibodies will inhibit human lymphocyte activation.

TABLE 2
INHIBITION OF MLC CO-CULTURES WITH NORMAL HUMAN IgM

| | Exp. #1* | Exp. #2* |
|---|---|---|
| PBL_{subject A} | 1169 | 1678 |
| PBL_{subject B} | 2594 | 2358 |
| PBL A + B_{control} | 5192 | 5914 |
| PBL A + B_{IgM ESRD**} | 659 | 1616 |

*Individuals in Exp #1 were different from individuals in Exp. #2.
**20 microgm of IgM was added to MLC cultures at initiation of the culture.

Non-Lytic Nature of IgM Anti-Lymphocyte Antibodies at 37° C.

About 40 to 60% cell lysis of normal lymphocytes was observed in presence of complement when the assay was performed at 15° C. Higher levels of cell lysis was observed with IL-2-activated lymphocytes, which have increased expression of receptors. Affinity purified IgM, when used at amounts of about 1.0 microgram or more, caused cell lysis, while CK15 lysed cells at concentrations of about 0.05 microgram or more. When the assay was performed at 37° C., however, less than about 5% lysis was observed with normal or IL-2 activated lymphocytes. These observations are in agreement with several reports clearly demonstrating that IgM anti-lymphocyte autoantibodies are lytic at colder temperatures but not at 37° C.

IgM Anti-Lymphocyte Antibodies Cause Cell Death of Lymphoma Cells at 37° C.

Malignant T lymphocytes, unlike normal IL-2 activated lymphocytes, undergo cell death in presence of IgM at 37° C. In these studies, we added 5 to 10 microgm of normal pooled IgM to 0.5×10⁶ Jurkat or Sup T-1 lymphocytes in 0.5 ml of RPM1 with 2% albumin. After thirty to 45 minutes incubation at 37° C. in 5% CO₂ cells were examined for cell death with Trypan Blue. No exogenous complement was added. Twenty to 35% of Jurkat or Sup T-1 cells were found to be dead under these conditions. There was less than 5% cell death of normal human lymphocytes or IL-2 activated lymphocytes when cultured under these conditions.

Effect of IgM on HIV-1 Infection of Cells

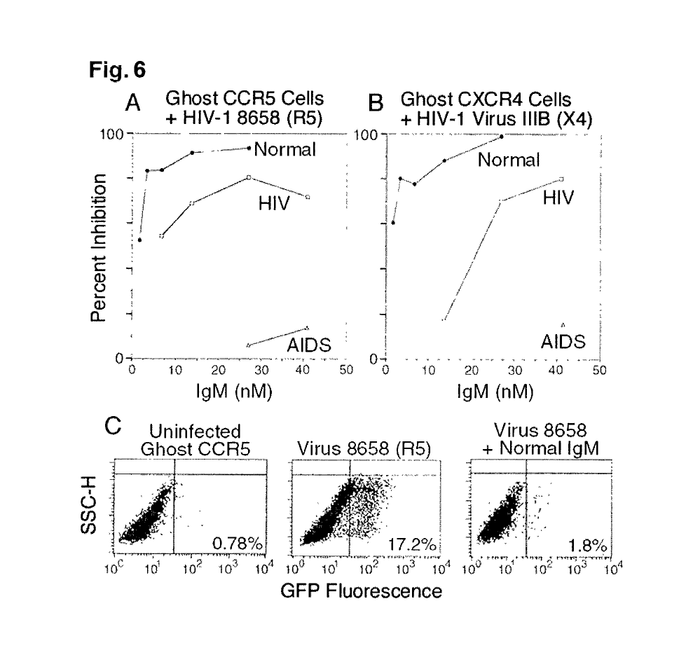

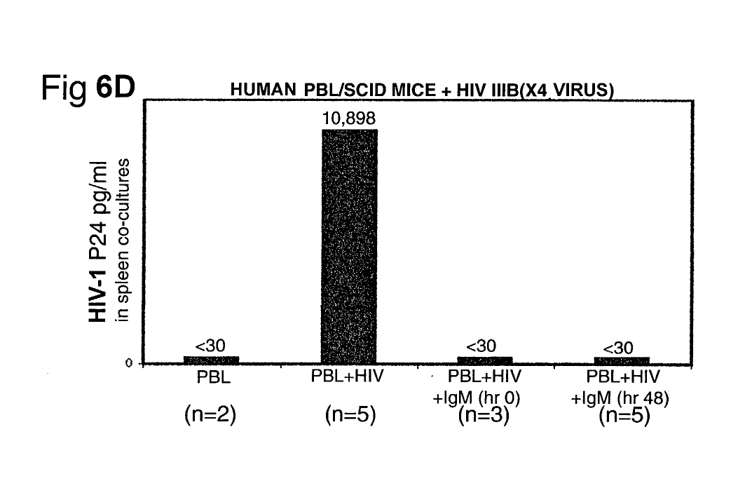

Studies were conducted to determine if IgM inhibited HIV-1 infectivity of cells bearing CCR5 or CXCR4 receptors. In these studies, we infected the CD4 positive GHOST CCR5 and GHOST CXCR4 transfected cell lines with HIV-1. A representative example from one of six experiments is depicted in FIGS. 6A, B and C. Normal pool IgM (including affinity purified Accurate IgM) and HIV IgM inhibited HIV-1 infectivity in nM concentrations. No or minimal inhibitory effects were noted with AIDS IgM even when used at higher concentrations. AIDS IgM did not enhance HIV-1 infectivity above baseline control levels (i.e. without IgM) indicating therefore that AIDS IgM did not potentiate viral infection or replication. Three experiments were performed to determine if normal IgM could restore the HIV inhibitory effect that was lacking in AIDS IgM. In all three experiments pooled Normal IgM when added to AIDS IgM (at equimolar concentrations) inhibited HIV-1 from infecting cells and the level of inhibition was similar to that observed when using Normal IgM alone suggesting therefore that the lack of inhibition seen with AIDS IgM is due to a deficiency of an IgM subset present in Normal IgM.

Similar observations were made using non-pooled IgM isolated from six individual normal sera and two HIV sera. Interestingly, the inhibitory effect of normal IgM varied when the IgM was used individually rather than pooled. IgM from three individuals significantly inhibited (>85%) viral strain R5 8442 while the same viral strain was partially inhibited (30-50%) with IgM from the other two individuals. However, IgM from only one of the three individuals that significantly inhibited R5 8442 and one of the two that partially inhibited R5 8442 significantly inhibited another HIV-1 strain i.e. R5 8658. Such observations would suggest that there are differences in normal IgM obtained from different individuals suggesting therefore that differences in epitope binding of virus and IgM antibody may influence degree of inhibition. Similar observations as with individual normal IgM were made with a murine monoclonal IgG antibody to CCR5 i.e. 2D7. Antibody 2D7 inhibited infectivity (by approximately 80%) of two of the three R5 viruses (i.e. 8397 and 8442 but not 8658) when used at 100 nM, again supporting the previously proposed concept that an antibody will inhibit viral infectivity of HIV-1 strains that interact with the same epitopes on the receptor as the antibody.

Individual differences in IgM mediated viral inhibition may also result from heterogeneity of human membrane receptors expressed on the viral envelope of the different R5 strains. To evaluate this possibility, different R5 viral strains were grown in PBL from the same individual that the IgM was obtained. In this one experiment, the differences in IgM mediated inhibitory activity with the different R5 viral strains persisted whether viruses were grown in autologous or allogeneic PBL.

Further experiments were performed to determine if the HIV-1 inhibitory effect was present in IgM that bound to lymphocytes. Hence, 0.5 ml of pooled normal human IgM(0.2 mg/ml) was absorbed with 80×10⁶ lymphocytes obtained from the blood of a normal individual. The lymphocytes, prior to the absorption procedure, were pre-cultured at 37° C. in 5% CO₂ for 24 hours to remove any serum IgM bound to the cell membrane. In both experiments, absorption of normal IgM with lymphocytes reduced by 70-80% the inhibitory effect of IgM on the infectivity of GHOST cells with the X4 virus IIIB and the R5 virus 8658, thus indicating that the observed inhibition of HIV-1 infectivity by IgM is mediated by IgM, which binds to the lymphocyte cell membrane and not to the virus. Additional support for such a conclusion comes from studies by other investigators clearly showing that normal human sera has no direct complement or antibody mediated lytic activity towards the HIV-1 virus.

Similar inhibitions of HIV-1 infectivity were observed using rat and rabbit IgM (purified from serum and obtained from Sigma). Animal IgM was more potent in inhibiting HIV-1 infectivity of cells in-vitro, when compared to human IgM.

Effect of IgM on HIV-1 Infection of human PBL/SCID Mice:

We performed these experiments to determine if IgM mediated inhibition of HIV-1 infectivity in-vitro could be reproduced in an in-vivo setting. Hence, human PBL were introduced into the peritoneum of SCID mice. The HIV-1×4 virus was injected intra peritoneally to infect the human PBL in this murine model. Human IgM from a single normal individual was injected intra peritoneally at the same time as the viral injection (at 0 hours) or 48 hours after the viral injection (at 48 hours).

Studies were not performed with HIV and AIDS IgM as it was difficult to obtain blood in quantities needed for these experiments. Data from one of two separate experiments with two different individual IgM and the same X4 virus III B are depicted in FIG. 6D. Data clearly indicates that normal IgM inhibits the III B virus from infecting human PBL in this in-vivo system. HIV-1 p24 antigen could not be detected in splenic co-cultures at three weeks even when IgM was administered to mice 48 hours after the HIV-1 infection. Furthermore, lack of detectable virus in IgM treated mice could not be attributed to a deficiency of human PBL in the spleen as the percent of human CD3 and CD4 positive cells in the spleen between IgM and non-IgM treated mice were similar. These data indicates that IgM inhibits HIV-1 replication in human PBL and the extent of in vivo inhibition was several fold more than what we observed in the GHOST cell system.

The results of the various studies, as discussed above, indicate that IgM purified from sera inhibits HIV-1 infectivity of cells. Purified IgM mediates inhibition of HIV-1 infectivity through binding of IgM to receptors important for HIV-1 entry into cells. Such receptors include, but are not limited to, CXCR4 and CCR5 chemokine receptors and the CD3 receptor. Potential mechanisms for inhibiting HIV-1 infectivity of cells include inhibition of HIV-1 cells entry through “blocking” of chemokine receptors and secondly through inhibition of cell activation by binding to CD3 or other lymphocyte receptors important for cell activation. IgM from normal sera has no direct anti-viral neutralizing effect and yet has the most inhibitory effect on HIV-1 infectivity. IgM purified from AIDS sera has minimal or no effect on HIV-1 infectivity, even though the AIDS IgM binds to Ghost cells and T cell lines and also enhances chemotaxis and cytosolic Ca⁺² induced by SDF-1. With respect to IgM binding and inhibition of HIV-1 infectivity, one plausible explanation for the observed difference between Normal IgM and HIV-1 IgM on the one hand and AIDS IgM on the other is that all these IgM preparations contain a heterogeneous group of IgM anti-lymphocyte antibodies except that AIDS IgM lacks the subset of IgM with HIV-1 inhibitory activity.

According to the present invention, IgM anti-lymphocyte autoantibodies limit the entry of the HIV-1 virus into cells and prolong the latency period because these antibodies bind to chemokine and other lymphocyte-surface receptors without lysing the cells at body temperature. The results shown herein indicate that disease progression to AIDS is associated with a marked reduction in the subset of IgM anti-lymphocyte autoantibodies that inhibit HIV-1 infectivity of cells.

Studies on IgM Anti-Lymphocyte Auto Antibodies Present in Kidney Transplant Recipients.

Since normal IgM inhibited the binding of chemokines (SDF-1 and RANTES) to their respective receptors and since ESRD IgM inhibited lymphocyte activation in a mixed lymphocyte culture (MLC), it became necessary to test whether in-vivo, there would be a strong correlation between the presence of high levels of these antibodies in the recipient and protection against kidney transplant rejections.

Accordingly, the level of IgM anti-lymphocyte antibody activity in the recipient was quantitated using flow cytometry to detect binding of IgM to donor lymphocytes (see FIG. 7A). Presence of high IgM binding to donor lymphocytes would also indicate that a similar level of IgM binding would occur with autologous leucocytes and donor endothelial cells.

Data in FIG. 7B clearly shows that the presence of low or high IgM anti-lymphocyte activity was clearly associated with significantly less rejections and less graft loss at one year. All patients in this study were given the same immunosuppressive agents.

According to the present invention, the inventor believes that IgM anti leucocyte antibodies mediate protection against rejections by binding to autologous leucocytes (thus inhibiting chemotaxis of leucocytes and lymphocyte activation) and receptors on donor endothelial cells. The inventor has prior art clearly demonstrating that certain kidney recipients have IgM in their serum that binds to both donor lymphocytes and kidney endothelial cells. These data are described in Lobo et al, Lancet 2: 879-83, 1980 and the material in this reference is incorporated herein by reference.

Discussion

While not wishing to be bound to any particular theory, there are several possible explanations for the entry of the HIV-1 virus into cells and increased viral replication despite the presence of a good level of IgM autoantibody to chemokine receptor during the asymptomatic state. One such explanation is the possibility that there exists a delicate balance between these low-affinity binding IgM antibodies and the viral load. Factors that predispose an individual to an increased viral load or that inhibit the B cells secreting IgM autoantibodies will lead to viral entry into cells and to disease progression. It is also possible that the recently described subset of B cells expressing CD4, CXCR4 and CCR5 receptors may be the same subset that secretes IgM autoantibodies. Over the years, this B cell subset could be exhausted or could be infected with HIV-1, thereby leading to a decrease in antibody production. Additionally, one cannot underscore the importance of other host factors (e.g., anti-viral IgG antibodies, chemokines and complement and cytotoxic T cells) that decrease the viral load. Perturbation in any of these host defense mechanisms could lead to an increased viral load.

Secondly, it is possible that in some HIV-1 infected individuals, IgM anti-lymphocyte antibody may only partially prevent entry of certain HIV-1 viral isolates, as indicated by some of the studies herein. This latter mechanism may provide another explanation for disease progression despite the presence of IgM anti-chemokine receptor autoantibodies.

That IgM autoantibodies inhibit HIV-1 virus from replicating supports the premise for a protective role mediated by these IgM anti-leukocyte antibodies. The use of human IgM anti-leukocyte antibodies to reduce HIV-1 infectivity (i.e., through receptor blockade and/or inactivation of cells) is an alternative approach for passive immunization, especially because it has been difficult to isolate human antibodies reactive to conserved neutralization epitopes on the HIV-1 virus. Receptor blockade employing IgM with reactivity to a broad range of chemokine and other receptors present on the lymphocytes may be particularly useful in situations where the HIV-1 virus switches its receptor usage, e.g., from CCR5 to CXCR4. Maintaining increased levels of such protective antibodies could also increase the latency period after HIV-1 infection. Additionally, it may be possible to design immunization strategies that enhance in-vivo IgM anti-lymphocyte NAA that are inhibitory to HIV-1 infectivity.

Inflammatory processes, angiogenesis and growth (and spread) of malignant cells are controlled by chemokines, cytokines, chemokine receptors and other receptors that activate (or inhibit) cell function. Such receptors are present on all leucocytes, endothelial cells and malignant cells. IgM anti-lymphocyte NAA, by binding to chemokine and other receptors (e.g. lipid rafts, CD3) could provide a regulatory role in the above-mentioned disorders or processes. The use of IgM anti-lymphocyte NAA, especially antibodies that inhibit chemokine receptor function or inhibit cell activation (i.e. with potential of causing apoptosis of malignant cells) would be particularly beneficial for inflammatory processes or growth and spread of malignant cells. Studies in renal transplant recipients clearly indicate that chemokines and chemokine receptors have a role in the rejection process. Data in this regard is reviewed in Hancock, W. W, J of Am Soc Nephrol 13: 821-824, 2002 and the material in this reference is incorporated herein by reference. Hence, the finding that kidney transplant recipients, with low or high levels of IgM anti lymphocyte antibodies, have no or minimal acute rejections would support the concept that IgM anti-lymphocyte antibodies inhibit chemokine receptor function and lymphocyte activation. One could employ passive immunization technique or alternatively design immunization strategies that specifically enhance in-vivo production of IgM anti-lymphocyte NAA (with inhibitory effect on chemokine receptor function or cell activation) to treat the various inflammatory processes and growth (and spread) of malignant cells.

The source of IgM antibodies may be heterologous, autologous or allogeneic. IgM antibodies with specificity for chemokine and other receptors on the leukocyte may be raised in vivo (i.e., in mice or other animals or in humans) or in vitro using cell culture techniques.

For example, IgM antibodies may be produced either in vivo or in vitro by genetic engineering whereby genes specific for IgM anti-lymphocyte antibodies are introduced into antibody-producing cells. These antibody-producing cells may then be introduced into an infected human or into immunodeficient animals where the cells produce IgM antibodies. In the alternative, these antibody-producing cells may be grown in vitro using hybridoma or other technology.

IgM antibodies with specificity for chemokine receptors may also be produced by isolating human or animal antibody-producing cells specific for IgM anti-lymphocyte antibodies and enhancing antibody production by such cells using hybridoma or other technology, including introduction of the cells into animals or humans. For example, human lymphocytes may be transplanted into immunodeficient mice, and the lymphocytes may then be stimulated with an agent that will activate B cells such as lipopolysaccharide (“LPS”)

Another method of producing IgM antibodies is by isolating human antibody-producing cells capable of generating human IgM from animals such as, for example, the XenoMouse™. IgM antibody production by such cells may then be enhanced in vitro employing hybridoma or other technology such as, for example, stimulating the isolated lymphocytes with LPS or other agent that will activate the cells, e.g., the EBV virus.

IgM antibodies may also be produced in vitro by isolating, from an individual, lymphocytes that can be then transformed with the EBV virus and introduced in a culture. A subset of these EBV transformed B lymphocytes will secrete IgM antibodies such that the resulting culture fluid contains these antibodies.

In addition, viruses, bacteria and other antigens (e.g., mitogens) may be used to stimulate B cells in vivo to generate IgM antibodies to leukocytes.

IgM antibodies produced outside an infected individual may be delivered to the individual by one of several routes of administration including, but not limited to, intravenous and intramuscular delivery.

IgG and IgA isotypes of naturally occurring autoantibodies (i.e. NAA) have also been described in prior art. The present invention also relates to IgG and IgA isotypes. All antibody isotypes (i.e. IgM, IgE, IgG and IgA) in this invention includes intact immunoglobulins or fragments of these antibodies. As such, throughout the specification and claims the use of the term “antibodies” or auto antibodies” includes naturally occurring antibodies of all isotypes used as intact immunoglobulins or fragments of these antibodies.

Having now fully described the invention with reference to certain representative embodiments and details, it will be apparent to one of ordinary skill in the art that changes and modifications can be made thereto without departing from the spirit or scope of the invention as set forth herein.

The material in the 11 references listed below is herein incorporated in this application to provide more detailed information that will enable the claims.

### REFERENCES

1. Olson T S and Ley K. Chemokines and chemokine receptors in leukocyte trafficking. Am J Physiol Regulatory Integrative Comp Physiol 283:R7-R28 (2002).

2. Gerard C and Rollins B J. Chemokines and disease. Nature Immunology 2 no 2: 108-115 (2001).

3. Onuffer J J and Horuk R. Chemokines, chemokine receptors and small-molecule antagonists: recent developments. TRENDS in Pharmacological Sciences 23 no 10:459-467 (2002).

4. Mellado M. et al. Chemokine signaling and functional responses: The role of receptor dimerization and TK Pathway Activation. Annu Rev Immunol 19:397-421 (2001).

5. Muller A. et al. Involvement of chemokine receptors in breast cancer metastasis. Nature 410: 50-56 (2001).

6. Werlen G and Palmer E. The TCR signalosome: a dynamic structure with expanding complexity. Current Opinion in Immunology 14:299-305 (2002).

7. Griggi T, Bauer R, Garofalo T, Kukel S, Lenti L, Massetti A P et al. Autoantibodies against ganglioside GM3 represents a portion of antilymphocytic antibodies in AIDS patients. Scan J of Immunol 1994;40:77-82.

8. Stimmler M M, Quismorio F P J, McGehee W G, Boylen T, Sharma O P. Anticardiolipin antibodies in acquired immunodeficiency syndrome. [see comments]. Archives of Internal Medicine 1989;149(8):1833-5.

9. Lacroix-Desmazes S, et al Selfreactive antibodies (natural autoantibodies) in healthy individuals J of Immunol Methods 216: 117-137, 1998.

10. Hancock W. W, Chemokines and Transplant Immunobiology J of Amer Soc Nephrol 13: 821-824, 2002.

11. Lobo PI et al. Cold lymphocytotaxins: and important cause of acute tubular necrosis occurring immediately after transplantation Lancet 2: 879-83, 1980.

What is claimed is:

1. A method of treating human diseases or disorders, comprising administering to the individual naturally occurring IgM antibodies (IgM NAA) having specificity to extracellular receptors present on lymphocytes.

2. The method of claim 1, wherein the receptors are chemokine receptors.

3. The method of claim 2, wherein the chemokine receptors are selected from the group consisting of CCR5, CXCR4, CCR2b, CCR3 and other chemokine receptors that have reactivity to naturally occurring IgM.

4. The method of claim 1, where in the receptors are non-chemokine receptors on lymphocytes and selected from the group of receptors that activate or inhibit cell function (or processes) or enhance death of cells or inhibit viral infectivity.

5. The method of claim 4, where in the receptor is the CD3 receptor or lipid rafts on the cell membranes.

6. The method of claim 1, wherein the receptors are leucocyte or endothelial cell or malignant cell surface receptors that are similar (but may not be identical) to those present on lymphocytes.

7. The method of claim 1, wherein the IgM antibodies having specificity to extracellular receptors present on lymphocytes are selected from the group consisting of human and animal naturally occurring IgM antibodies (referred to as IgM NAA).

8. The method of claim 7, wherein the IgM NAA can be selected from monoclonal or polyclonal NAA or synthetic or recombinant IgM NAA or antibody fragments of IgM NAA having specificity to extracellular receptors present on leucocytes.

9. The method of claim 1, wherein the human disease or disorder comprises virus mediated disease, autoimmune disease, inflammatory states and cellular malignancies.

10. The method of claim 9, wherein the viral mediated disease is HIV-1 or other viral diseases in which virus cell entry involves chemokine receptors that bind to IgM NAA and/or other leucocyte receptors.

11. The method of claim 9, wherein the autoimmune disease is selected from the group of systemic lupus erythematosus, rheumatoid arthritis, vasculitis and other autoimmune conditions in which chemokines, leucocytes and other leucocyte and endothelial cell receptors that bind to IgM NAA mediate the disorder.

12. The method of claim 9 wherein the inflammatory state is selected from the group of asthma, sarcoidosis, atherogenesis and atherosclerosis or allograft and xenograft rejections in which chemokines, leucocytes and other leucocyte and endothelial cell receptors that bind to IgM NAA mediate the disorder.

13. The method of claim 9, wherein the cellular malignancy involves lymphoid or non-lymphoid malignancies and where chemokines, chemokine receptors and other non-chemokine receptors that bind to IgM NAA enhance tumor growth and spread (or metastases) and cell death.

14. The method of claim 1, wherein therapy would comprise administering IgM NAA to inhibit progression of disease processes or prevent disease processes.

15. The method of claim 10, wherein IgM anti-lymphocyte NAA binds to receptors targeted by the virus and/or binds to other extracellular leucocyte receptors important in enhancing viral infectivity of cells.

16. The method of claims 11, 12, or 13 where in the IgM anti-leucocyte NAA binds to chemokine and non-chemokine receptors present on leucocytes, endothelial cells and malignant cells.

17. The method of claim 1 or 14, wherein the IgM anti-lymphocyte NAA are administered to the individual by oral routes, by subcutaneous routes, intravenously or intramuscularly or their production is enhanced in-vivo with one or more agents elected from the group consisting of viruses, inactive bacteria, antigens and mitogens.

18. A method of producing IgM anti-leucocyte NAA for treating human diseases or disorders, comprising introducing genes specific for IgM anti-leucocyte NAA into antibody-producing cells and producing the IgM anti-leucocyte antibodies in vitro or in vivo.

19. A method of producing animal or human IgM anti-leucocyte NAA for treating human diseases or disorders, comprising isolating human or animal antibody producing cells and enhancing production of IgM NAA in-vitro by the antibody producing cells.

20. A method of producing IgM anti-leucocyte NAA, comprising isolating human antibody-producing cells from animals capable of generating human IgM and enhancing production of IgM anti-leucocyte NAA in vitro or in vivo by the antibody-producing cells.

21. The method of claim 18, 19, or 20, wherein the production of IgM anti-leucocyte NAA by the antibody-producing cells is enhanced using hybridoma technology or cell culture techniques.

22. The method of claim 18, 19 or 20, wherein the production of IgM anti-leucocyte NAA by the antibody-producing cells is enhanced using viruses, bacteria or antigens.

23. A method of producing IgM anti-leucocyte NAA in vivo comprising injecting one or more individuals with one or more elected from the group consisting of viruses, inactive bacteria, viral and bacterial products, fungal products, plant antigens and mitogens, wherein the IgM antibodies are used to treat viral infections, auto-immune diseases, inflammatory states and cellular malignancies.

24. A method of treating virus mediated disease, human autoimmune diseases, inflammatory states and cellular malignancies in an individual comprising administering to the individual IgM anti-leucocyte NAA produced according to claims 18, 19, 20, 21, 22 or 23.
